(19) United States
(12) Patent Application Publication
(10) Pub. No.: US 2022/0050310 A1
(43) Pub. Date: Feb. 17, 2022

(54) Auto Tint Safety Goggle Apparatus

(71) Applicant: Samina Henry, Moreno Valley, CA (US)

(72) Inventor: Samina Henry, Moreno Valley, CA (US)

(21) Appl. No.: 16/990,415

(22) Filed: Aug. 11, 2020

(51) Int. Cl. G02C 7/10; G02C 7/08; G02C 11/08; G02C 11/04

(52) U.S. Cl. CPC G02C 7/102; G02C 11/04; G02C 11/08; G02C 7/086

(57) ABSTRACT

An auto tint safety goggle apparatus for use during all lighting conditions without changes lenses includes a frame body having a frame top side, a frame left side, a frame right side, and a frame bottom side defining a frame opening. A multi-layer lens is coupled to the frame body and conforms to the frame opening. The multi-layer lens comprises an outer transparent layer, an inner transparent layer, and a photochromatic layer coupled between the outer transparent layer and the inner transparent layer. The photochromatic layer reacts to light to selectively darken the multi-layer lens. A pair of loops is coupled to the frame body. The pair of loops is coupled to the frame left side and the frame right side. A strap is coupled to the pair of loops. The strap is elasticized and has an adjustable length.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to safety goggle devices and more particularly pertains to a new safety goggle device for use during all lighting conditions without changes lenses.

### (2) Description of Related Art Including Information Disclosed Under 37 CFR 1.97 and 1.98

The prior art relates to safety goggle devices. Most existing devices have removable lenses and require the user to change different lenses for different lighting conditions. Existing devices also lack both a photochromatic film layer, an anti-fog film layer, and scratch resistance.

### BRIEF SUMMARY OF THE INVENTION

An embodiment of the disclosure meets the needs presented above by generally comprising a frame body having a frame top side, a frame left side, a frame right side, and a frame bottom side defining a frame opening. A multi-layer lens is coupled to the frame body and conforms to the frame opening. The multi-layer lens comprises an outer transparent layer, an inner transparent layer, and a photochromatic layer coupled between the outer transparent layer and the inner transparent layer. The photochromatic layer reacts to light to selectively darken the multi-layer lens. A pair of loops is coupled to the frame body. The pair of loops is coupled to the frame left side and the frame right side. A strap is coupled to the pair of loops. The strap is elasticized and has an adjustable length.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an auto tint safety goggle apparatus according to an embodiment of the disclosure.

FIG. 2 is a front elevation view of an embodiment of the disclosure.

FIG. 3 is a rear elevation view of an embodiment of the disclosure.

FIG. 4 is a side elevation view of an embodiment of the disclosure.

FIG. 5 is a cross-sectional view of an embodiment of the disclosure along the line 5-5 of FIG. 2.

### DETAILED DESCRIPTION OF THE INVENTION

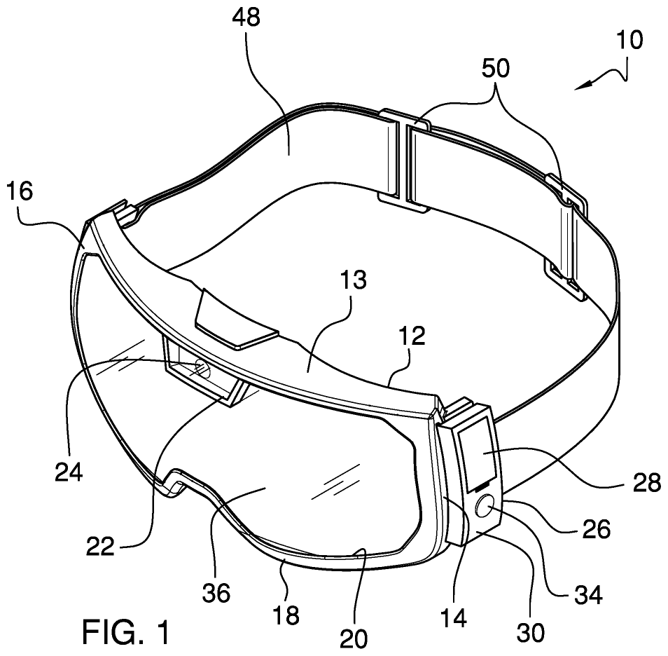

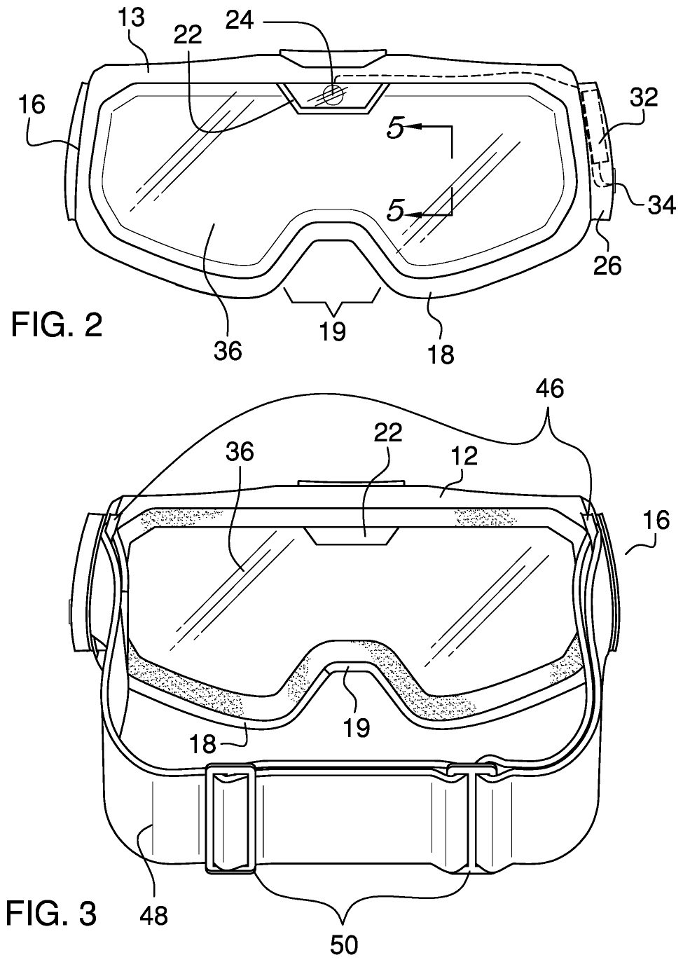

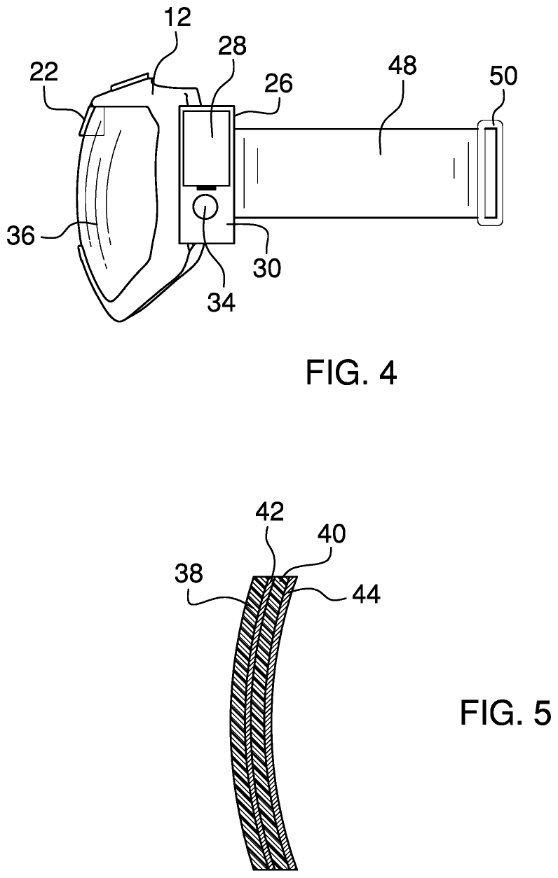

With reference now to the drawings, and in particular to FIGS. 1 through 5 thereof, a new safety goggle device embodying the principles and concepts of an embodiment of the disclosure and generally designated by the reference numeral 10 will be described.

As best illustrated in FIGS. 1 through 5, the auto tint safety goggle apparatus 10 generally comprises a frame body 12 having a frame top side 13, a frame left side 14, a frame right side 16, and a frame bottom side 18 defining a frame opening 20. The frame bottom side 18 may have a medial nose portion 19 to accommodate a wearer's nose.

A light housing 22 is coupled to frame body 12. The light housing 22 may be coupled to the frame top side 14. The light housing 22 may be trapezoidal and may extend into the frame opening 20.

A light 24 is coupled within the light housing 22. The light 24 is adjusted to a brightness and wavelength for use at night without dilating pupils or interfering with night vision goggles. The light 24 may produce red light. A power housing 26 is coupled to the frame body 12. The power housing 26 may be coupled to the frame left side 14. The power housing 26 may have a selectively removable battery cover 28 coupled to a housing outer face 30. A battery 32 is coupled within the power housing 26 and is in operational communication with the light 24. A power button 34 is coupled to the power housing 26. The power button 34 is coupled to the housing outer face 30 and is in operational communication with the light 24.

A multi-layer lens 36 is coupled to the frame body 12. The multi-layer lens 36 may be arcuate and conforms to the frame opening 20. The multi-layer lens 36 comprises an outer transparent layer 38 and an inner transparent layer 40. A photochromatic layer 42 is coupled between the outer transparent layer 38 and the inner transparent layer 40. The outer transparent layer 38 may be scratch resistant. The photochromatic layer 42 reacts to light to selectively darken the multi-layer lens 36 to allow for use in all lighting conditions without swapping lenses. Each of the outer transparent layer 38 and the inner transparent layer 40 may be, but is not limited to, a durable plastic material such as polycarbonate. An anti-fog layer 44 may be continuously coupled to the inner transparent layer 40 to prevent the multi-layer lens 36 from becoming fogged and obstructing the wearer's view.

A pair of loops 46 is coupled to the frame body 12. The pair of loops 46 is coupled to the frame left side 14 and the frame right side 16. A strap 48 is coupled to the pair of loops 46. The strap 48 is elasticized and has an adjustable length. The strap 48 may have a pair of adjustment buckles 50.

In use, the apparatus is worn like a traditional pair of goggles. In bright light the photochromatic layer 42 tints the multi-layer lens 36 to protect the wearer's eyes from glare. As the external light lessens, the multi-layer lens 36 becomes progressively more transparent. In dark or low-light settings, the user may activate the light 24 using the power button 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An auto tint safety goggle apparatus comprising:
a frame body having a frame top side, a frame left side, a frame right side, and a frame bottom side defining a frame opening;
a multi-layer lens coupled to the frame body, the multi-layer lens conforming to the frame opening, the multi-layer lens comprising:
an outer transparent layer;
an inner transparent layer; and
a photochromatic layer coupled between the outer transparent layer and the inner transparent layer, the photochromatic layer reacting to light to selectively darken the multi-layer lens;
a pair of loops coupled to the frame body, the pair of loops being coupled to the frame left side and the frame right side; and
a strap coupled to the pair of loops, the strap being elasticized and having an adjustable length.

2. The auto tint safety goggle apparatus of claim 1 further comprising the multi-layer lens further comprising an anti-fog layer continuously coupled to the inner transparent layer.

3. The auto tint safety goggle apparatus of claim 1 further comprising a light housing coupled to frame body; a light coupled within the light housing; a power housing coupled to the frame body; a battery coupled within the power housing, the battery being in operational communication with the light; and a power button coupled to the power housing, the power button being in operational communication with the light.

4. The auto tint safety goggle apparatus of claim 3 further comprising the light housing being coupled to the frame top side, the light housing being trapezoidal and extending into the frame opening.

5. The auto tint safety goggle apparatus of claim 3 further comprising the power housing being coupled to the frame left side, the power housing having a selectively removable battery cover coupled to a housing outer face; the power button being coupled to the housing outer face.

6. The auto tint safety goggle apparatus of claim 1 further comprising the multi-layer lens being arcuate.

7. The auto tint safety goggle apparatus of claim 1 further comprising the strap having a pair of adjustment buckles.

8. An auto tint safety goggle apparatus comprising:
a frame body having a frame top side, a frame left side, a frame right side, and a frame bottom side defining a frame opening;
a light housing coupled to frame body, the light housing being coupled to the frame top side, the light housing being trapezoidal and extending into the frame opening;
a light coupled within the light housing;
a power housing coupled to the frame body, the power housing being coupled to the frame left side, the power housing having a selectively removable battery cover coupled to a housing outer face;
a battery coupled within the power housing, the battery being in operational communication with the light;
a power button coupled to the power housing, the power button being coupled to the housing outer face and being in operational communication with the light;
a multi-layer lens coupled to the frame body, the multi-layer lens being arcuate and conforming to the frame opening, the multi-layer lens comprising:
an outer transparent layer;
an inner transparent layer;
a photochromatic layer coupled between the outer transparent layer and the inner transparent layer, the photochromatic layer reacting to light to selectively darken the multi-layer lens; and
an anti-fog layer continuously coupled to the inner transparent layer;
a pair of loops coupled to the frame body, the pair of loops being coupled to the frame left side and the frame right side; and
a strap coupled to the pair of loops, the strap having a pair of adjustment buckles, the strap being elasticized and having an adjustable length.
